(19) United States
(12) Patent Application Publication
(10) Pub. No.: US 2024/0003799 A1
(43) Pub. Date: Jan. 4, 2024

(54) METHOD OF ACQUISITION AND ANALYSIS OF COAGULATION HAEMOSTATIC PARAMETERS OF A BLOOD SAMPLE

(71) Applicant: VISCOP S.r.l., Roma (IT)

(72) Inventors: Marco RANUCCI, Roma (IT); Francesco NAPOLITANO, Roma (IT); Franco SCORZIELLO, Roma (IT)

(73) Assignee: VISCOP S.r.l., Roma (IT)

(21) Appl. No.: 18/254,894

(22) PCT Filed: Nov. 29, 2021
(86) PCT No.: PCT/IB2021/061064

§ 371 Date: May 30, 2023

(30) Foreign Application Priority Data
Nov. 30, 2020 (IT) 102020000029126

(51) Int. Cl. G01N 11/14; G01N 33/49

(52) U.S. Cl. CPC G01N 11/142; G01N 33/4905

(57) ABSTRACT

A method of acquisition and analysis of coagulation haemostatic parameters of a blood sample comprises: supply of at least one blood sample; preparation of at least one viscometer in contact with the blood sample; acquisition of a plurality of viscosity data of the blood sample during a coagulation haemostatic process; and calculation of at least one characteristic parameter of the coagulation haemostatic process depending on the density values measured, the parameter being selected from: time to gel point, maximum clot viscosity and steady clot viscosity.

### TECHNICAL FIELD

The present invention relates to a method of acquisition and analysis of coagulation haemostatic parameters of a blood sample.

### BACKGROUND ART

For some time now, in the medical field, diagnostic studies have been carried out on the viscous-elastic characteristics of the blood, and in particular on the relevant variations during the coagulation phase of the haemostatic process.

The coagulation rate and clot stability of a blood sample depends on multiple factors related to the patient's clinical picture and directly related to the activity of the coagulation system, to the platelet function, to fibrinolysis, and to a multiplicity of other factors influenced by genetic factors, diseases and drug intake.

To date, the measurement of clot viscosity and elasticity is carried out by several methods which explore hemodynamic processes during the coagulation phase.

In general, these methods require the addition of agents which stimulate the haemostatic-coagulation process, such as e.g. kaolin, tissue factor, and others.

In this case, however, the haemostatic-coagulation process is measured under static conditions, in this case the blood sample is subjected to rotation at a shear rate value not corresponding to a shear rate value existing physiologically at any point of the body circulation.

In detail, a first known method consists in the so-called thromboelastography (TEG).

Thromboelastography involves transferring a sample of blood taken from the patient into a rotating container containing sensor means which are adapted to detect changes in the resistance and elasticity of the blood.

In the absence of activating factors (native TEG), the process takes an extremely long time, thus preventing its implementation in clinical practice.

For this reason, the blood sample is activated by means of kaolin, or alternatively, by the combination of kaolin with tissue factor (rapid-TEG).

The sensor means are operatively connected to processing means for the processing of the detected viscosity values in variable graphic representations depending on the specific needs of the operators in the field.

In particular, the blood sample is subjected to rotation at a preset shear rate value substantially equal to 0.5 sec⁻¹; this value is not similar to the corresponding physiological shear rate to which the blood in the blood vessels is subjected, thus decreasing the veracity of the analysis.

Moreover, this first method has some drawbacks among which we have to include the fact that it provides arbitrary units of measurement such as e.g. the millimeter, which is not comparable to experimental data of viscosity expressed according to the International System in Poiseuille and, therefore, not very plausible to reality.

An alternative method is the so-called ReoRox in which the vessel containing the blood sample is subjected to free oscillation and the sensor means separately detect changes in the elasticity and viscosity of the sample itself In the aforementioned method, the shear rate value to which the blood is subjected is likely to the physiological values of the blood compared to previous methods but it is, nevertheless, preset and unchangeable.

Also in this case the units of measurement with which the detected data are expressed and represented in graphs are arbitrary and do not allow for a direct comparison with other experimental viscosity data.

A second known method consists in the so-called thromboelastometry (ROTEM).

Similarly to thromboelastography (TEG), thromboelastometry (ROTEM) also requires that the blood sample is contained in a container having sensor means operatively connected to the processing means of the collected data. Similarly to thrombolestography, there are different activators, such as kaolin, for the INTEM method, or tissue factor for the EXTEM method.

In this case, the sensor means are driven in rotation until they are slowed down by blood coagulation.

Therefore, data is collected as a function of the slowing of the rotation of the sensor means as the clot is formed. The processing means read and process this slowdown by graphically translating it into a curve.

However, even in this case, thromboelastometry (ROTEM) measures changes in clot elasticity and resistance during the haemostatic phase, thus providing data expressed in millimeters and therefore not comparable to experimental viscosity measurements.

In addition, the presence of artificial activators generates a thrombin burst which causes platelet activation regardless of the presence of platelet inhibitory drugs such as, e.g., aspirin, thienopyridines, ticagrelor, which are very common in clinical practice.

To overcome this drawback, complex and laborious TEG thromboelastography techniques (platelet mapping) are required.

### DESCRIPTION OF THE INVENTION

The main aim of the present invention is to devise a method of acquisition and analysis of coagulation haemostatic parameters of a blood sample which allows carrying out measurements expressed in units of measurement directly comparable to experimental viscosity data.

One object of the present invention is to devise a method of acquisition and analysis of coagulation haemostatic parameters of a blood sample which allows dynamic and continuous exploration of the coagulation haemostatic process. Another object of the present invention is to devise a method of acquisition and analysis of coagulation haemostatic parameters of a blood sample C which simulates physiological blood activation, thus avoiding the use of activators and thus allowing an assessment of the platelet function as well.

A further object of the present invention is to devise a method of acquisition and analysis of coagulation haemostatic parameters of a blood sample which allows the mentioned drawbacks of the prior art to be overcome within a simple, rational, easy and effective to use as well as affordable solution.

The aforementioned objects are achieved by the present method of acquisition and analysis of coagulation haemostatic parameters of a blood sample having the characteristics of claim 1.

### BRIEF DESCRIPTION OF THE DRAWINGS

Other characteristics and advantages of the present invention will become more apparent from the description of a preferred, but not exclusive, embodiment of a method of acquisition and analysis of coagulation haemostatic parameters of a blood sample C, illustrated by way of an indicative, yet non-limiting example, in the accompanying tables of drawings wherein:

FIG. 1 is a schematic representation of the viscometer according to the method according to the invention;

FIGS. 2-6 are time-dependent graphs representative of the haemostatic coagulation process;

FIGS. 7 and 8 are electron microscope images corresponding to specific haemostatic-coagulation parameters.

### EMBODIMENTS OF THE INVENTION

The method of acquisition and analysis of coagulation haemostatic parameters of a blood sample C comprises at least the following phases of:
supply of at least one blood sample C;
preparation of at least one viscometer 1 in contact with the blood sample C;
acquisition of a plurality of viscosity data of the blood sample C during a coagulation haemostatic process;
calculation of at least one characteristic parameter of the coagulation haemostatic process, wherein the parameter is selected from: time to gel point (TGP), maximum clot viscosity (MCV) and steady clot viscosity (SCV).

The blood sample C is native, i.e. it is free of artificial activators.

It cannot, however, be ruled out from the scope of the present disclosure that the blood sample may be treated with activators or inhibitors in order to carry out specific analyses related to the presence of heparin, to the platelet function (MCV) and to the contribution of fibrinogen to SCV.

By way of a non-limiting example, such activators/inhibitors comprise: heparinases, GPIIbIIIa receptor inhibitors, reptilase, ADP.

The viscometer 1 comprises a contact portion 2 operable in rotation around a relevant axis 3 and a supporting surface 4 positioned below the contact portion 2, wherein the contact portion 2 is configured to contact the supporting surface 4.

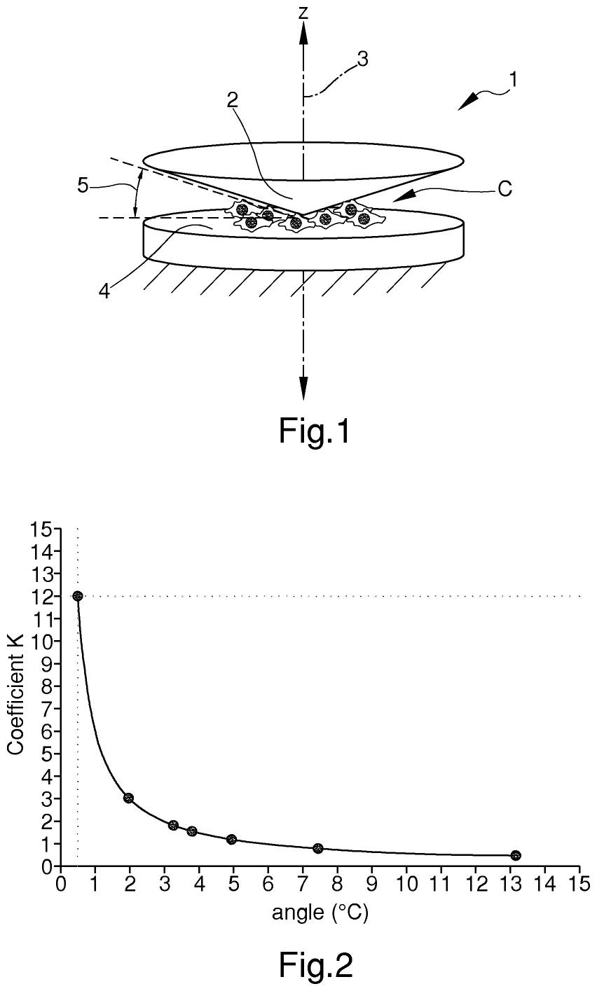

As visible in FIG. 1, the contact portion 2 has a conical conformation provided with a vertex configured to contact the surface of the blood sample C.

In the present case, the vertex defines, with the surface of the blood sample C, an angle 5 having a predefined angular amplitude.

Preferably, the angle 5 has an amplitude comprised between 0.3° and 1°.

According to a preferred embodiment of the method according to the invention, the angle 5 has an amplitude substantially equal to 0.5°.

Furthermore, the supporting surface 4 has a slab-like conformation, i.e., in which the dimensions of length and width are preponderant over thickness.

The supporting surface 4 is free of surface irregularities, i.e., it is smooth.

In addition, it is of paramount importance that the supporting surface 4 is inert, i.e., does not cause the activation of the haemostatic-coagulation process of the blood sample C.

Advantageously, the supporting surface 4 is disposable; this means that, for each type of blood sample C analyzed, it is necessary to replace the supporting surface 4 with a new one.

Preferably, the supporting surface 4 is made of a material selected from: graphite and aluminum.

It cannot, however, be ruled out from the scope of the present disclosure that the supporting surface 4 is made of a durable, i.e., non-disposable, material in which the supporting surface 4 can be wiped clean following its use.

Advantageously, the blood sample C is deposited on the supporting surface 4 in a quantity comprised between 300 μL and 400 μL.

Preferably, the blood sample C is deposited on the supporting surface 4 in a quantity equal to 360 μL.

The acquisition phase is performed by means of processing means such as e.g. plc, microcontroller, pc and the like integrated or operatively connected to the viscometer 1.

Prior to the phase of supply of the blood sample C, the method comprises a phase of calibrating the viscometer 1 comprising at least the following steps:
operation in rotation of the contact portion 2, the latter being moved away from the supporting surface 4;
moving the contact portion 2 closer to the supporting surface 4 until they are brought in contact with each other;
measurement and acquisition of the viscosity values;
moving the contact portion 2 away from the supporting surface 4 as far as a predefined distance from the latter.

Advantageously, the predefined distance corresponds to a detected viscosity value comprised between 0.5 cP and 3 cP.

According to a preferred embodiment of the method according to the invention, the predefined distance corresponds to a detected viscosity value comprised between 1 cP and 2 cP.

Following the calibration phase of the viscometer 1, the blood sample C is supplied and the viscometer 1 is placed in contact with the blood sample C itself.

At this point, the method comprises a phase of operation in rotation of the contact portion 2 at a predefined speed V adapted to apply to the surface of the blood sample C a shear rate calculated using the following formula: shear rate (sec⁻¹)=V (rpm)×k wherein k=5.95×A⁻¹⁰⁰⁸.

It is specified that in the present disclosure the expressions “cutting speed” and “shear rate” will be used interchangeably with each other.

In detail, the shear rate corresponds to the deformation rate of the blood sample C during the haemostatic-coagulation process.

In this regard, it must be stressed that, in a Newtonian fluid, the shear rate is directly proportional to the deformation rate and the proportionality constant between them is the dynamic viscosity; the latter is a thermo-physical property of the fluid, regardless of the deformation rate.

On the contrary, in a non-Newtonian fluid like blood, the proportionality between the applied shear rate and the deformation rate of the fluid is not linear. In this context, the identification of a speed of rotation of the contact portion 2 equal to the physiological shear rate of a medium-sized vein is crucial for the implementation of the method according to the present invention to be precise and accurate.

This means that the blood sample C in the implementation of the method according to the invention is subjected to a physiological shear rate, i.e. comparable to the shear rate to which the blood is subjected during the body circulation in a medium-sized vein.

For this purpose, the shear rate applied to the blood sample C is equal to 240 sec⁻¹ .

Preferably, the speed of rotation of the contact portion 2 is comprised between 15 rpm and 25 rpm.

According to a preferred embodiment of the method according to the invention, the speed of rotation of the contact portion 2 is equal to 20 rpm.

At this point, the method comprises a phase of graphic processing of the viscosity values detected during the haemostatic-coagulation process.

The graphic representation comprises the step of processing a time-dependent curve of the detected viscosity values, wherein the time to gel point, the maximum clot viscosity and the steady clot viscosity are identified on such a curve.

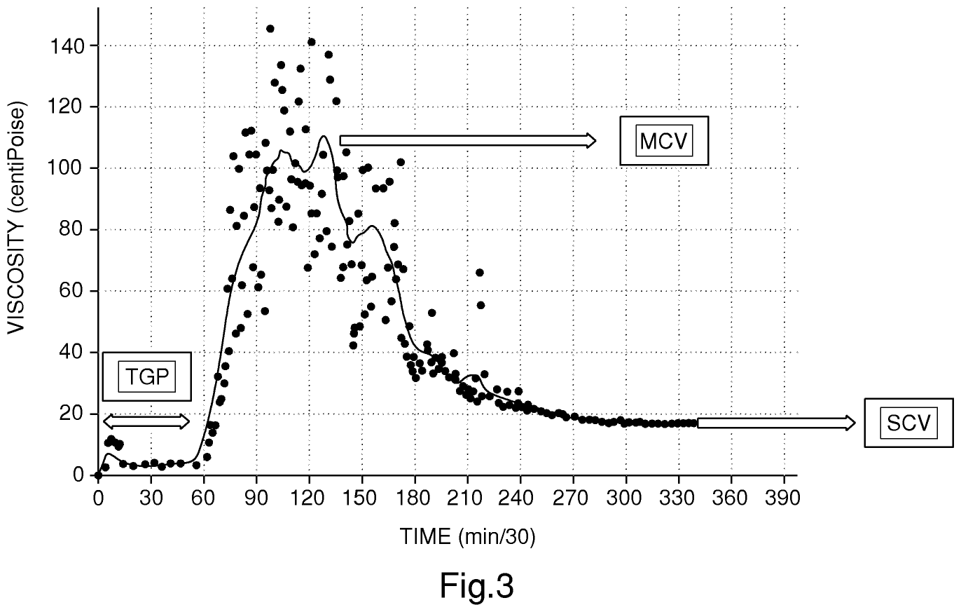

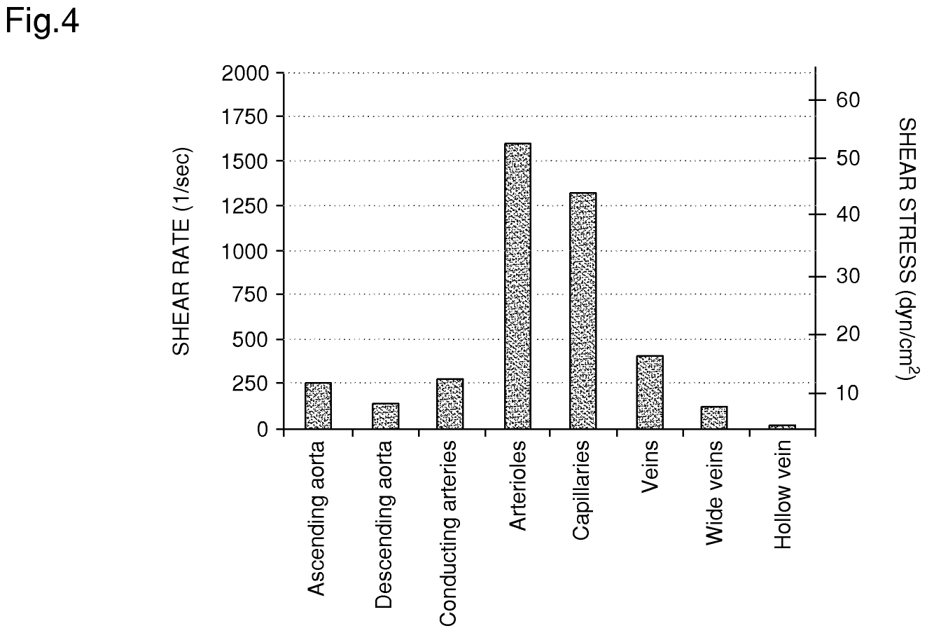

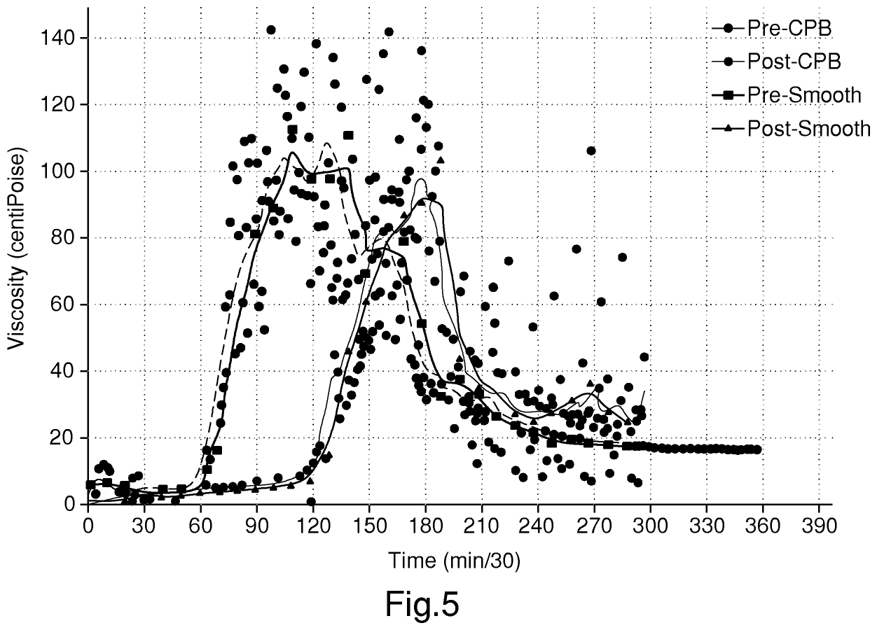

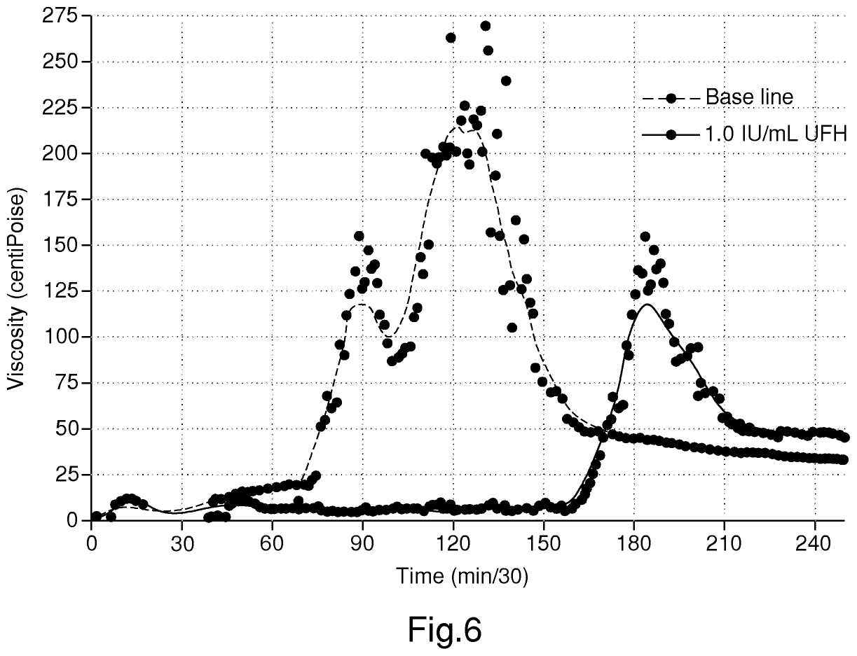

In detail, as shown in FIGS. 3 and 4, the aforementioned curve is representative of the haemostatic-coagulation phenomenon in which the above mentioned parameters are clearly identifiable, i.e. time to gel point (TGP), maximum clot viscosity (MCV) and steady clot viscosity (SCV).

The graphic representation of the curve is carried out by means of suitable processing means such as e.g. a plc, microcontroller, pc and the like built in or operatively connected to the viscometer 1.

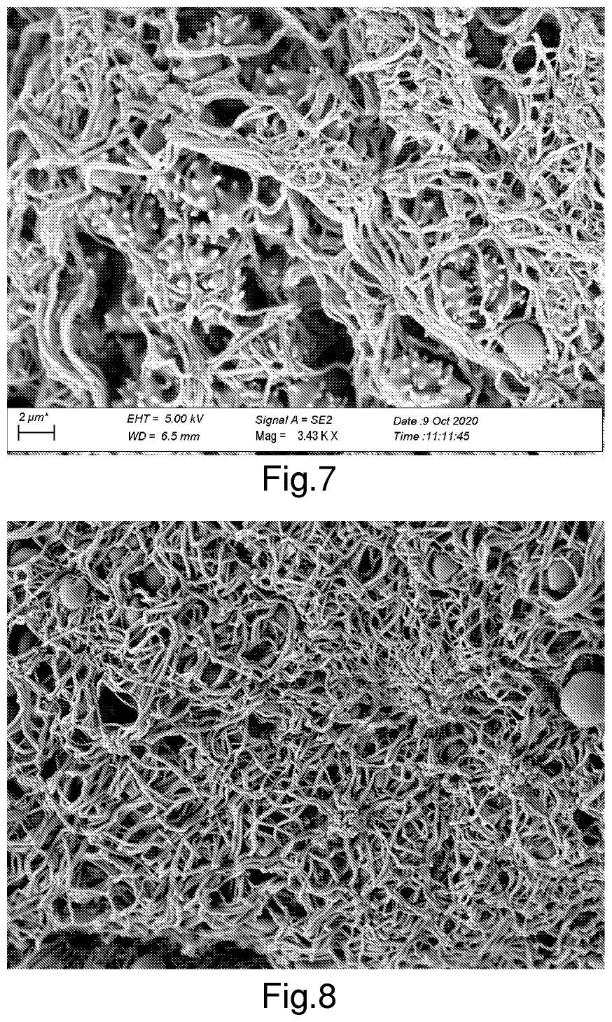

It should be stressed that these parameters are crucial in the dynamic study of the haemostatic-coagulation process. As can be seen in FIG. 7, the interpretation of the curve obtained (FIGS. 4 and 7) and in particular of what is physiologically observed at MCV and SCV allows displaying the fibrin and platelet component of the clot by means of electron microscopy analysis.

For example, FIG. 7 shows the phase of maximal platelet activation and the formation of an unorganized fibrin network.

At the same time, FIG. 8 shows a stabilized fibro-platelet network corresponding to SCV.

It has in practice been ascertained that the described invention achieves the intended objects.

The fact is emphasized that the particular expedient of providing a contact portion configured to contact the blood sample at a predefined distance and configured to apply on the latter a shear rate, which is also predefined, allows measures expressed in units of measurement directly comparable to experimental viscosity data, thus exploring the coagulation haemostatic process in a dynamic and continuous manner.

1. A method of acquisition and analysis of coagulation haemostatic parameters of a blood sample, said method comprises at least the following phases of:
supply of at least one blood sample;
preparation of at least one viscometer in contact with said blood sample;
acquisition of a plurality of viscosity data of said blood sample during a coagulation haemostatic process; a
calculation of at least one characteristic parameter of said coagulation haemostatic process depending on said density values measured, said parameter being selected from: time to gel point, maximum clot viscosity and steady clot viscosity.

2. The method according to claim 1, wherein said viscometer comprises at least one contact portion operable in rotation around a relevant axis and at least one supporting surface positioned below said contact portion, said contact portion being configured to contact said supporting surface.

3. The method according to claim 2, wherein said contact portion has a conical conformation provided with a vertex configured to contact the surface of said blood sample, said vertex defining with said surface of said blood sample an angle having a predefined angular amplitude.

4. The method according to claim 2, further comprising: a phase of operation in rotation of said contact portion at a predefined speed adapted to apply to said surface of said blood sample a cutting speed calculated using the following formula: cutting speed=V×k where k=5.95×A^{-1.008}.

5. The method according to claim 3, wherein said angle has an amplitude comprised between 0.3° and 1°.

6. The method according to claim 2, further comprising: a calibration phase of said viscometer, said calibration phase being previous to said phase of supply and comprises at least the following steps:
operation in rotation of said contact portion, the latter being moved away from said supporting surface;
moving said contact portion closer to said supporting surface until they are brought in contact with each other;
measurement and acquisition of said viscosity values; and
moving said contact portion away from said supporting surface as far as a predefined distance said supporting surface

7. The method according to claim 6, wherein said predefined distance corresponds to a detected viscosity value comprised between 0.5 cP and 3 cP.

8. The method according to claim 1, further comprising: a phase of graphic processing of said viscosity values detected during the haemostatic-coagulation process, said graphic processing comprising the step of processing a time-dependent curve of said viscosity values detected, wherein said time to gel point, said maximum clot viscosity and said steady clot viscosity are identified on said curve.

9. The method according claim 1, wherein said blood sample is deposited on said supporting surface in a quantity comprised between 300 μLand 400 μL.

10. The method according to claim 2, wherein said speed of rotation of said contact portion is comprised between 15 rpm and 25 rpm.

11. A method of acquisition and analysis of coagulation haemostatic parameters of a blood sample, said method comprising:
supplying at least one blood sample;
preparing at least one viscometer in contact with said blood sample;
acquiring a plurality of viscosity data of said blood sample during a coagulation haemostatic process; and
calculating at least one characteristic parameter of said coagulation haemostatic process depending on said density values measured, wherein
said parameter being selected from: time to gel point, maximum clot viscosity and steady clot viscosity.
